(19) United States
(12) Patent Application Publication
(10) Pub. No.: US 2006/0009832 A1
(43) Pub. Date: Jan. 12, 2006

(54) Balloon catheter and method and system for securing a stent to a balloon catheter

(75) Inventor: Beau M. Fisher, Danville, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/172,181

(22) Filed: Jun. 30, 2005

Related U.S. Application Data
(60) Provisional application No. 60/586,510, filed on Jul. 9, 2004.

(51) Int. Cl. A61F 2/06

(52) U.S. Cl. 623/1.11

(57) ABSTRACT

A stent is retained on a balloon catheter with a stent retention feature in the form of a pillow which minimizes sliding of the stent on the balloon during stent delivery and deployment. The balloon catheter/stent assembly includes a balloon catheter having an elongated catheter shaft and a balloon positioned at a distal end of the elongated catheter shaft. An inflation lumen extends within the elongated catheter shaft for inflation and deflation of the balloon. The stent is mounted and crimped onto the balloon and at least one pillow is formed on the balloon adjacent the ends of the stent. The at least one pillow is formed from the balloon and has an outer diameter larger than the outer diameter of the mounted and crimped stent.

### RELATED APPLICATIONS

This application claims priority to U.S. Provisional Patent Application Ser. No. 60/586,510, filed Jul. 9, 2004, the entire contents of which are incorporated herein by reference.

### BACKGROUND

The present invention relates to tissue-supporting medical devices and delivery systems. More particularly the invention relates to a balloon catheter and to a method and system for securing a stent to a balloon catheter.

Stents are expandable cylindrical devices that are implanted within a bodily lumen of a living animal or human to support the organ and maintain patency. Stents are typically introduced percutaneously, and transported transluminally until positioned at a desired location. The stent is then expanded either mechanically, generally by the expansion of a balloon positioned inside the stent to support the lumen. Once expanded within the lumen the stents become encapsulated within the body tissue. Stents can be biodegradable or remain a permanent implant.

Known stent designs include monofilament wire coil stents (U.S. Pat. No. 4,969,458); welded metal cages (U.S. Pat. Nos. 4,733,665 and 4,776,337); and, most prominently, thin-walled metal cylinders with axial slots formed around the circumference (U.S. Pat. Nos. 4,733,665, 4,739,762, and 4,776,337). Known construction materials for use in stents include polymers, organic fabrics, biocompatible metals, such as, stainless steel, gold, silver, tantalum, cobalt alloys, titanium, and shape memory alloys such as Nitinol, and biodegradable polymers and metal alloys.

U.S. Pat. Nos. 4,733,665; 4,739,762; 4,776,337; 6,241,762; and 6,562,065 disclose expandable and deformable stents in the form of thin-walled tubular members with axial slots allowing the members to be expanded radially outwardly by a balloon into contact with a body passageway.

Generally stents are delivered after dilation of a lumen by percutaneous transluminal angioplasty (PTA) or atherectomy. When stents are delivered without a predilation process, the process is called direct stenting. With either type of procedure it is important to retain the stent on the balloon catheter while the catheter is advanced through the body lumen to the location where the stent will be implanted. If the stent moves on the balloon or is dislodged from the balloon it may not be accurately delivered to the lumen. It is also important that, after expansion, the stent is no longer adhered to the balloon.

The systems for securing stents onto balloons for delivery include systems for crimping or compressing the stent onto the balloon to achieve adherence. Crimping is often used in combination with another technique to increase adherence. For example, stent securing systems including adhesives are described in U.S. Pat. Nos. 6,682,553 and 6,635,078. Other systems for improving adherence involve deformation of the balloon beneath the stent to extend portions of the balloon between cells in the stent creating improved retention. Examples of such balloon deformation systems are described in U.S. Pat. Nos. 6,309,402 and 6,666,880.

However, there is a need to retain stents more securely on a balloon catheter without using adhesives that may create difficulties in releasing the stent from the balloon and without adversely affecting any drug on the stent.

### SUMMARY OF THE INVENTION

The present invention relates to a balloon catheter with a pillow and a method and system for securing a stent to a balloon catheter.

In accordance with one aspect of the invention, a balloon catheter/stent assembly comprises a balloon catheter having an elongated catheter shaft, a balloon positioned at a distal end of the elongated catheter shaft, and an inflation lumen extending within the elongated catheter shaft for inflation and deflation of the balloon; a stent mounted and crimped onto the balloon, the mounted and crimped stent having an outer diameter; and at least one pillow on the catheter balloon, the at least one pillow formed from the balloon and having an outer diameter larger than the outer diameter of the mounted and crimped stent, wherein the at least one pillow is formed adjacent to an end of the stent.

In accordance with another aspect of the invention, a system for securing a stent to a balloon catheter comprises a restrainer having a restrainer lumen configured to receive a balloon catheter with a stent crimped thereon, the restrainer having a first inner diameter which is approximately equal to an outer diameter of the crimped stent and a second inner diameter which is greater than an outer diameter of the crimped stent providing a step within the lumen of the restrainer; a catheter pressuring device for pressurizing the balloon within the restrainer to form at least one pillow adjacent to an end of the stent, the at least one pillow having an outer diameter larger than the outer diameter of the crimped stent; and a heating device for heating the at least one pillow to set the pillow shape.

In accordance with an additional aspect of the invention, a method of forming a catheter/stent assembly comprises the steps of crimping a stent onto a balloon of a balloon catheter; positioning at least a portion of the crimped stent and balloon into a restrainer having a first inner diameter which is approximately equal to an outer diameter of the crimped stent and a second inner diameter which is greater than an outer diameter, wherein the portion of the restrainer with the first inner diameter is placed around the stent and the portion of the restrainer with the second inner diameter is positioned adjacent an end of the stent; pressurizing the balloon within the restrainer to form at least one pillow adjacent to an end of the stent, the at least one pillow having an outer diameter larger than the outer diameter of the crimped stent; and heating the at least one pillow to set the pillow shape.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a side cross sectional view of a distal end of a stent delivery system including a balloon catheter and a stent, with the balloon catheter in a deflated condition and the stent in an unexpanded condition.

FIG. 2 is a side cross sectional view of the stent delivery system of FIG. 1 with the balloon catheter in an inflated condition and the stent in an expanded condition.

FIG. 3 is a side cross sectional view of a restrainer configured with the stent delivery system received in the lumen of the restrainer for formation of stent retaining pillows.

FIG. 4 is a schematic perspective view of a system for securing a stent to a balloon catheter.

### DETAILED DESCRIPTION

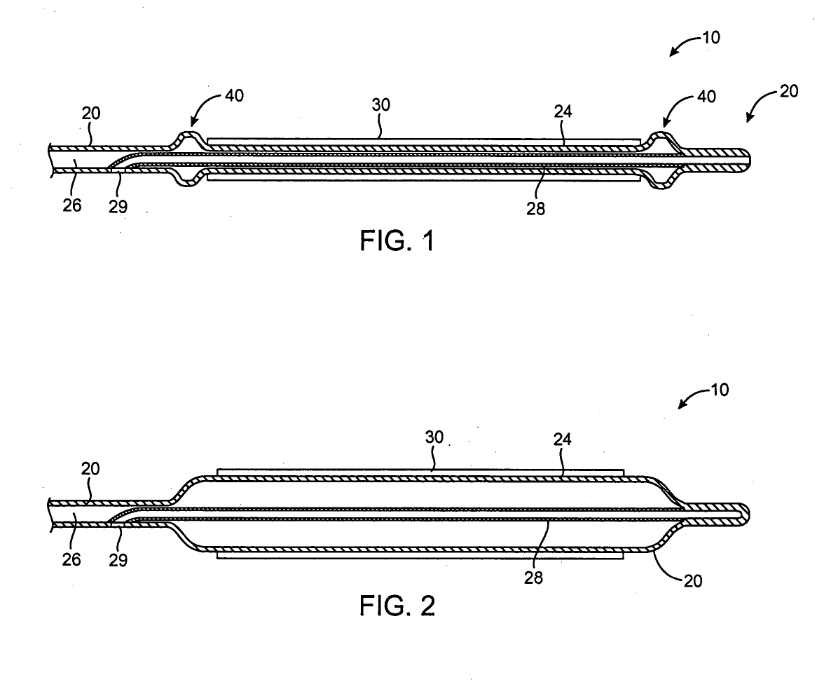

FIG. 1 illustrates a catheter/stent apparatus 10 including a balloon catheter 20 with a stent 30 mounted thereon. The balloon catheter 20 has an elongated catheter shaft 22, only a portion of which is shown. A balloon 24 is positioned at a distal end of the elongated catheter shaft 22 and an inflation lumen 26 extends within the elongated catheter shaft for inflation and deflation of the balloon.

The stent 30 which is mounted and crimped onto the balloon 24 may be any known stent, for example, the stent can be a drug delivery stent such as those illustrated in U.S. patent Publication No. 2003/0199970, published on Oct. 23, 2003, which is incorporated herein by reference in its entirety. The stent 30 is positioned on the balloon 24 with a small portion of the balloon extending beyond each end of the stent. This extension of the balloon 24 beyond the end of the stent 30 helps to ensure complete expansion of the stent.

The catheter can be any of the known angioplasty or stent delivery catheters. As shown in FIGS. 1 and 2, the catheter 20 is preferably a rapid exchange catheter having a short guidewire lumen 28 beneath the balloon 24 and a proximal guidewire opening 29 which is closer to the balloon than to the proximal end of the catheter.

As shown in FIG. 1, at least one and preferably two pillows 40 are provided on the catheter balloon 24 between the ends of the stent 30 and the ends of the balloon. The pillows 40 are formed from the material of the expandable balloon 24 and are formed by expansion and heat setting the balloon after mounting and crimping the stent on the balloon. The pillows 40 extend radially outward from the outer surface of the uninflated balloon 24 to help to retain the stent 30 onto the balloon during stent insertion. The pillows 40 have an outer diameter which is larger than the outer diameter of the mounted and crimped stent 30. When the pillows 40 are formed adjacent to the ends of the stent 30 they improve stent retention over crimping alone.

As shown in FIG. 2, when the balloon 24 is expanded, the pillows 40 expand to essentially the same diameter as the remainder of the balloon 24 and essentially disappear.

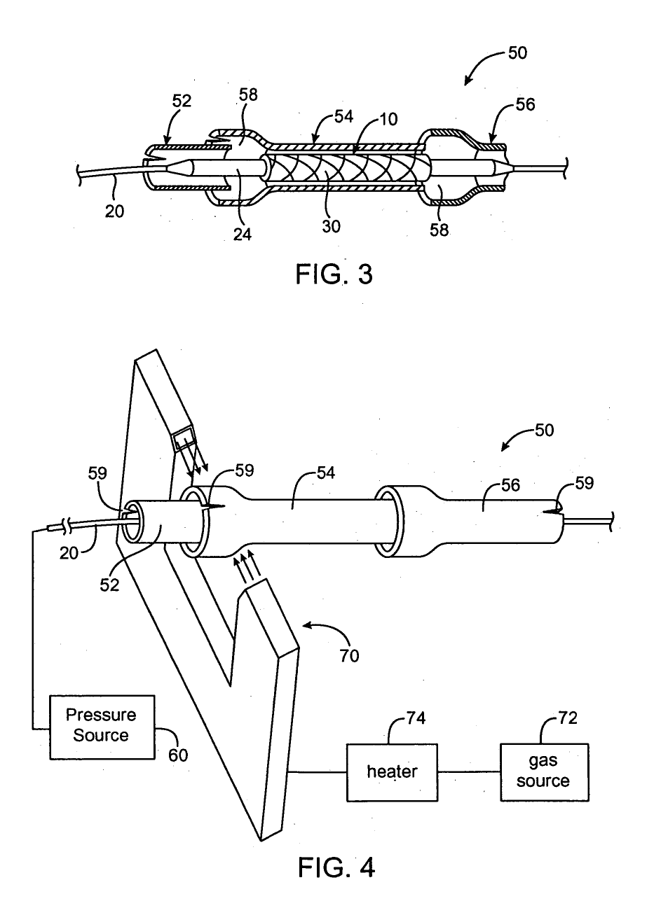

FIG. 3 shows a cut-away view of a system of sheaths which form a restrainer 50 over the catheter/stent assembly 10 for the pillowing process. The sheaths include a proximal sheath 52, a center sheath 54, and a distal sheath 56. The sheaths are assembled over the catheter/stent assembly 10 to form gaps 58 at the ends of the stent 30. Heat and pressure of the pillowing process will form the balloon 24 into these gaps to create the pillows 40 or small lumps that prevent the stent from sliding on the balloon 24 during use.

The proximal sheath 52 is a simple substantially cylindrical sheath having an inner diameter which is sufficiently large to fit over the catheter shaft and a portion of the end of the balloon 24 without the stent 30. The center sheath 54 is stepped with a first inner diameter which is approximately equal to an outer diameter of the crimped stent 30 and a second larger inner diameter which is greater than an outer diameter of the stent providing a step within the lumen of the sheath. The distal sheath 56 is also stepped with a first inner diameter which is the same as that of the proximal sheath 52 and a second larger diameter which is the same as the larger diameter of the center sheath 54. The sheaths are each slid over the catheter 20 and stent 30 and assembled in a manner such that the gaps 58 are formed precisely adjacent the ends of the stent for formation of the pillows 40.

The gaps 58 and the pillows which are formed in these gaps can have a length of about 0.5 to about 5.0 mm, preferably about 1 to about 2.5 mm. The gaps 58 are preferably formed to create a pillow having a height above the maximum crimped outer diameter of the stent which is approximately the same as the thickness of the stent wall. More particularly, the height which the pillow extends beyond the outer diameter of the crimped stent is about ½ to about 3 times the thickness of the stent wall. A difference between an outer diameter of the crimped stent and an outer diameter of the pillow can be about 0.01 to about 1.0 mm, preferably about 0.05 to about 0.3 mm, and more preferably about 0.1 to about 0.2 mm.

Each of the sheaths 52, 54, 56 can be provided with one or more slits 59 or perforations, i.e. two slits on one end of each sheath. The slits 59 allow the sheaths to be peeled off of the catheter/stent assembly 10 after formation of the pillows 40.

The multiple sheath system is used to allow the sheaths to be easily positioned over the catheter. Alternative sheath systems can also be used to form a restrainer, such as a single sheath with two steps or multiple sheaths that have been positioned partially coaxially to form the steps. One example of a multiple sheath system includes three small diameter sheaths placed with two of the sheaths proximal and distal of the pillow locations and a central sheath extending the length of the stent so that gaps occur between the small diameter sheaths at the locations of the pillows. A large diameter sheath can then be placed over the three small diameter sheaths to form the gaps into which the balloon is expanded to form the pillows.

As shown in FIG. 4, once the sheaths have been positioned over the catheter/stent assembly 10 the pillows can be expanded and set. The catheter is connected to a pressure source 60 for pressurization of the balloon. A pressure source 60 or pressure manifold provides a source of pressurized fluid. The pressurization of the balloon depends on the balloon material, construction, and size. According to one embodiment, the catheter is pressurized to between about 100 and about 200 psi, preferably about 110 to about 160 psi. The pressure is selected to be a pressure at which the balloon is expanded into the gaps 58 and is pressed into contact with the inner diameter of the sheaths to form the pillows 40.

The catheter is maintained pressurized while the pillows are heat set. The heat set process applies heat to the pillow areas of the catheter, without causing significant heating of the stent. Particularly in the mounting of a drug delivery stent, heating of the stent could cause degradation of the drug or melting of any polymer drug carrier. The application of heat to the pillow can be performed in many ways. A hot gas jet 70 can be used as the heating device. The hot gas jet 70 aimed at the area to be heated provides heating of the pillow 40 without overheating the stent. In one example of a heating device, a gas source 72, such as nitrogen, air, or inert gas, provides gas to a heater 74, which heats the gas to be delivered by the hot gas jet 70. The hot gas jet can be integrated into the heater as a hot box. The hot gas jet 70 shown in FIG. 4 directs the hot gas at the catheter pillow area from two opposite directions. Other configurations of hot gas jets may include a single gas outlet or more than two gas outlets. The hot gas may be applied through a porous holding device, such as a foam pad.

The sheath system for retaining the catheter/stent assembly 10 during the pillowing procedure and for providing the gaps 58 into which the balloon expands to form the pillow is only one example of the type of retainer which can be used. Another retainer can include a mold which can be closed around the catheter/stent assembly and provides the gaps 58 for formation of the pillows. An adjustable mold can be used to accommodate different size stents.

The heating device including the hot gas jet 70 is only one example of the type of heating devices that can be used. Other heating devices such as a heating block can also be used. As long as the heat is concentrated at the area in which the pillow is to be formed, cooling of the adjacent stent area can be avoided. Shorter heating times also minimize any overheating of the stent. For example, the pillow areas can each be heated for about 20 to about 100 seconds, preferably about 25 to about 45 seconds. The hot gas temperature will be selected depending on the balloon material used. For example, a hot gas temperature of at least 175 degrees F., and preferably about 200 degrees F. is selected for a pillowing temperature for commonly used balloon of nylon or nylon-like materials.

Following the heating step, the formed pillows can be cooled by a cold jet or other cooling system while the internal catheter pressure is retained. Upon cooling, the catheter is removed from the pressure source or manifold and the sheaths are removed.

The final pillow configuration having a pillow diameter greater than a stent crimped diameter significantly improves retention of the stent 30 on the catheter 20. This improved retention is due to the fact that the stent 30 would have to expand beyond its crimped diameter to pass over the pillows 40. It has been found that pillows 40 extending out from the outer diameter of the stent a distance which is approximately equal to the thickness of the stent walls do not have any noticeable affect on the ability of the balloon catheter to deliver the stent to a target site through narrowed vessels. In fact, the balloon pillows 40 are sufficiently pliable that they compress during stent delivery when the catheter passes through tight spaces.

The stent according to the present invention can be used for supporting a variety of ducts or lumens within the body, such as coronary or peripheral arteries, lumens of the tracheal or bronchial tree, billiary ducts, and the like.

While the invention has been described in detail with reference to the preferred embodiments thereof, it will be apparent to one skilled in the art that various changes and modifications can be made and equivalents employed, without departing from the present invention.

1. A balloon catheter/stent assembly comprising:
a balloon catheter having an elongated catheter shaft, a balloon positioned at a distal end of the elongated catheter shaft, and an inflation lumen extending within the elongated catheter shaft for inflation and deflation of the balloon;
a stent mounted and crimped onto the balloon, the mounted and crimped stent having an outer diameter; and
at least one pillow on the catheter balloon, the at least one pillow formed from the balloon and having an outer diameter larger than the outer diameter of the mounted and crimped stent, wherein the at least one pillow is formed adjacent to an end of the stent.

2. The assembly of claim 1, wherein a difference between the outer diameter of the mounted and crimped stent and the outer diameter of the at least one pillow is at least 0.05 mm.

3. The assembly of claim 1, wherein a difference between the outer diameter of the mounted and crimped stent and the outer diameter of the at least one pillow is about a thickness of the stent or greater.

4. The assembly of claim 1, wherein the at least one pillow has a length of about 0.5 mm to about 5 mm.

5. The assembly of claim 1, wherein the at least one pillow comprises two pillows, with one pillow adjacent each opposite end of the stent.

6. The assembly of claim 1, wherein the at least one pillow comprises a continuous portion of the balloon which has a larger diameter than a remainder of the balloon created by pressurization and heat setting.

7. The assembly of claim 1, wherein the at least one pillow is compliant and compresses to pass through tight lesions.

8. The assembly of claim 1, wherein the at least one pillow prevents the stent from moving longitudinally on the balloon prior to expansion of the stent and allows the stent to move longitudinally on the balloon upon expansion of the stent.

9. The assembly of claim 1, wherein the balloon catheter is a rapid exchange catheter.

10. A method of direct stenting comprising using the balloon catheter/stent assembly of claim 1 to stent a coronary artery without predilation.

11. A system for securing a stent to a balloon catheter, the system comprising:
a restrainer having a restrainer lumen configured to receive a balloon catheter with a stent crimped thereon, the restrainer having a first inner diameter which is approximately equal to an outer diameter of the crimped stent and a second inner diameter which is greater than an outer diameter of the crimped stent providing a step within the lumen of the restrainer;
a catheter pressuring device for pressurizing the balloon within the restrainer to form at least one pillow adjacent to an end of the stent, the at least one pillow having an outer diameter larger than the outer diameter of the crimped stent; and
a heating device for heating the at least one pillow to set the pillow shape.

12. The system of claim 11, wherein the restrainer comprises a first sheath providing the step within a lumen of the sheath.

13. The system of claim 12, wherein the restrainer further comprises a second sheath having a step within a lumen and a largest inner diameter substantially the same as the second inner diameter of the first sheath.

14. The system of claim 11, wherein the restrainer includes a die providing the step within the die.

15. The system of claim 11, wherein the heating device is a hot gas nozzle configured to direct hot gas at the at least one pillow without substantially heating the crimped stent.

16. The system of claim 11, wherein the heating device heats the at least one pillow to a temperature of at least 175 degrees F.

17. The system of claim 11, wherein the catheter pressurizing device pressurizes the catheter to about 110 to 160 psi.

18. The system of claim 11, wherein the restrainer is configured to create a pillow having an outer diameter which is at least 0.05 mm larger than the crimped stent.

19. The system of claim 11, wherein the restrainer is configured to create a pillow having a length of about 0.5 mm to about 5 mm.

20. The system of claim 11, wherein the at least one pillow comprises two pillows, with one pillow adjacent each opposite end of the stent.

21. A method of forming a catheter/stent assembly comprising:
crimping a stent onto a balloon of a balloon catheter;
positioning at least a portion of the crimped stent and balloon into a restrainer having a first inner diameter which is approximately equal to an outer diameter of the crimped stent and a second inner diameter which is greater than an outer diameter, wherein the portion of the restrainer with the first inner diameter is placed around the stent and the portion of the restrainer with the second inner diameter is positioned adjacent an end of the stent;
pressurizing the balloon within the restrainer to form at least one pillow adjacent to an end of the stent, the at least one pillow having an outer diameter larger than the outer diameter of the crimped stent; and
heating the at least one pillow to set the pillow shape.

22. The method of claim 21, wherein the restrainer comprises a first sheath providing a step within a lumen of the sheath.

23. The method of claim 22, wherein the restrainer further comprises a second sheath having a step within a lumen and inner diameters substantially the same as the first sheath.

24. The method of claim 21, wherein the restrainer includes a die providing the step within the die.

25. The method of claim 21, wherein the heating step is performed by a hot gas nozzle configured to direct hot gas at the at least one pillow without substantially heating the crimped stent.

26. The method of claim 21, wherein the heating step comprises heating the at least one pillow to a temperature of at least 175 degrees F.

27. The method of claim 21, wherein the pressurizing step comprises pressurizing the catheter to about 110 to 160 psi.

28. The method of claim 21, wherein the pillow has an outer diameter which is at least 0.05 mm larger than the crimped stent.

29. The method of claim 21, wherein the restrainer is configured to create a pillow having a length of about 0.5 mm to about 5 mm.

30. The method of claim 21, wherein the at least one pillow comprises two pillows, with one pillow formed adjacent each opposite end of the stent.

31. The method of claim 21, further comprising removing the stent delivery system and stent from the restrainer.

32. The method of claim 21, further comprising cooling the at least one pillow prior to removing the stent delivery system and stent from the restrainer.
